(19) United States
(12) Patent Application Publication
(10) Pub. No.: US 2018/0362667 A1
(43) Pub. Date: Dec. 20, 2018

(54) MONOCLONAL ANTIBODY SPECIFIC TO PCV2 AND METHOD FOR DIAGNOSING PMWS USING SAME

(71) Applicants: BIOPOA, INC., Yongin-si (KR); SEOUL NATIONAL UNIVERSITY R&DB FOUNDATION, Seoul (KR)

(72) Inventors: Sun Hee Cho, Seongnam-si (KR); Tae Eun Kim, Yongin-si (KR); Jun Ho Chung, Seongnam-si (KR); Hyo Ri Kim, Seoul (KR); Jun Yeong Jin, Gwacheon-si (KR)

(21) Appl. No.: 15/520,894

(22) PCT Filed: May 20, 2015
(86) PCT No.: PCT/KR2015/005065

§ 371 Date: Apr. 21, 2017

(30) Foreign Application Priority Data
Oct. 21, 2014 (KR) 10-2014-0142442

(51) Int. Cl. C07K 16/46; C07K 16/08; G01N 33/532

(52) U.S. Cl. CPC C07K 16/46; C07K 16/081; G01N 2800/26; C07K 2317/622; C07K 2317/24; G01N 33/532

(57) ABSTRACT

The present invention relates to a monoclonal antibody specific to porcine circovirus 2 (PCV2) and a method for diagnosing post-weaning multi-systemic wasting syndrome (PMWS) using the same. More specifically, the present invention relates to monoclonal antibodies C4-1 and C4-8 of scFV-human Cκ fusion recombinant protein, which specifically binds to a decoy epitope of porcine circovirus 2, and to a method for diagnosing post-weaning multi-systemic wasting syndrome using the same. The monoclonal antibody of the present invention makes it possible to determine whether an antibody against PCV2 is a neutralizing antibody by a vaccine antigen or an antibody induced by immune decoy.

### BACKGROUND OF THE INVENTION

### Field of the Invention

The present invention relates to a monoclonal antibody specific to porcine circovirus 2 (PCV2) and a method for diagnosing post-weaning multi-systemic wasting syndrome (PMWS) using the same. More specifically, the present invention relates to monoclonal antibodies C4-1 and C4-8 of an scFV-human Cκ fusion recombinant protein, which specifically binds to a decoy epitope of porcine circovirus 2, and a method for diagnosing post-weaning multi-systemic wasting syndrome (PMWS) using the same.

### Related Art

Porcine circovirus 2 (PCV2) is a small non-enveloped icosahedral virus classified into the family of Circoviridae, which contains single-stranded circular DNG genome of about 1.76 kb and has two major open reading frames (ORFs). ORF1 produces viral replication protein (Rep) and ORF2 produces capsid protein (CP) (J. Gillespie et al. J Vet Intern Med, 2009, 23, 1151-6, Porcine circovirus type 2 and porcine circovirus-associated disease). With regard to the capsid protein (CP), which is produced by the transcription of ORF2 of PCV2, a set of 3 forms a single surface and an icosahedral structure formed of 20 surfaces is established and thereby a structure of virus-like particle (VLP) is completed. The region of amino acid residues positioned from the 169^{th} to the 180^{th} forming each CP subunit is known as a strong epitope. The epitope [CP (169 to 180)] is buried inside in the VLP structure and not exposed to the outside and thus it is difficult for antibody to access to the epitope and thus has a low correlation with a neutralizing antibody of virus.

When PCV2 VLP, which is produced by CP expressed in Baculovirus, was immunized, high neutralizing antibody was produced but anti-CP (169 to 180) antibody was not produced well. In contrast, when CP monomers were immunized or in pigs in the PMWS state by being infected with PCV2, there was a trend that the neutralizing antibody was formed at a low level but the anti-CP (169 to 180) antibody was produced at a high level, thus suggesting that the CP (169 to 180) epitope plays the role of immune decoy (Trible B R et al., J Virol. 2012 86(24), 13508-13514, Recognition of the different structural forms of the capsid protein determines the outcome following infection with porcine circovirus type 2). Accordingly, in the development of a PCV2 vaccine, it is necessary to use an antigen, in which VLP is well formed, as a vaccine so that the neutralizing antibody against PCV2 is formed at a high level while the anti-CP (169 to 180) antibody is not well formed so that the CP (169 to 180) residues are not exposed to the outside in the vaccine antigen for PCV2.

Meanwhile, as the methods for detecting PCV2 antibody, immunoperoxidase monolayer assay (IPMA) and ELISA using PCV2 virus particle and recombinant CP have been developed and used (Pileri E et al., Vet J. 2014, 201(3), 429-32, Comparison of the immunoperoxidase monolayer assay and three commercial ELISAs for detection of antibodies against porcine circovirus type 2). However, it is difficult to accurately diagnose PCV2 simply based on the antibody titer because the replication of PCV2 is inhibited and a higher level of the neutralizing antibody is exhibited in pigs vaccine with a PCV2 VLP vaccine, whereas the titer of the antibody, which cannot neutralized PCV2, especially the antibody at the C-terminus of CP, is elevated (Trible B R et al., Vaccine 2012(30) 4079-85, Antibody responses following vaccination versus infection in a porcine circovirus-type 2 (PCV2) disease model show distinct differences in virus neutralization and epitope recognition).

Accordingly, in the diagnosis of PCV2 antibody, it is necessary to distinguish the PCV2 antibody not only with respect to its titer but also with respect to whether the PCV2 antibody is a neutralizing antibody or an antibody of the region associated with the immune decoy at the C-terminus of CP, which has a low correlation with virus neutralization.

### SUMMARY OF THE INVENTION

The present invention provides a monoclonal antibody specific to porcine circovirus 2 (PCV2) and a method for diagnosing post-weaning multi-systemic wasting syndrome (PMWS) using the same.

In an aspect, a monoclonal antibody specific to porcine circovirus 2 (PCV2) is disclosed.

With respect to the monoclonal antibody according to the present invention, the monoclonal antibody may be an scFV-human Cκ fusion recombinant protein which specifically binds to a decoy epitope of PCV2.

With respect to the monoclonal antibody according to the present invention, the ScFV-human Cκ fusion recombinant protein, which specifically binds to the decoy epitope of PCV2, may be C4-1, C4-8, or C4-1 and C4-8.

With respect to the monoclonal antibody according to the present invention, the decoy epitope of the PCV2 may be 12 amino acids positioned from the 169^{th} to the 180^{th}.

With respect to the monoclonal antibody according to the present invention, the amino acids may be STIDYFQPMMKR.

In another aspect, a reagent for diagnosing post-weaning multi-systemic wasting syndrome (PMWS), which includes an antigen for diagnosis, a monoclonal antibody for capturing the antigen for diagnosis, a label for detection, a monoclonal antibody to which the label for detection is bound, and a reagent for measuring the activity of the label for detection, is disclosed.

With respect to the reagent for diagnosis according to the present invention, the monoclonal antibody for capturing the antigen for diagnosis may be an scFV-human Cκ fusion recombinant protein which specifically binds to a decoy epitope of porcine circovirus 2 (PCV2).

With respect to the reagent for diagnosis according to the present invention, the ScFV-human Cκ fusion recombinant protein, which specifically binds to the decoy epitope of PCV2, may be C4-1, C4-8, or C4-1 and C4-8.

In still another aspect, a method for analyzing the characteristics of porcine circovirus 2 (PCV2) antibody using an enzyme-linked immunosorbent assay (ELISA) is disclosed. The method may include:

performing a competitive reaction between the monoclonal antibody according to an aspect of the present invention and the antibody in the serum of a subject infected with PCV2 or vaccinated subject;

measuring the absorbance of the monoclonal antibody; and

determining whether the antibody in the serum is a neutralizing antibody or an antibody induced by immune decoy based on the absorbance of the monoclonal antibody.

With respect to the method for analyzing the characteristics of PCV2 antibody, the method may further include determining the antibody as a neutralizing antibody when the absorbance of the monoclonal antibody is constantly maintained.

With respect to the method for analyzing the characteristics of PCV2 antibody according to the present invention, the method may further include determining the antibody as an antibody induced by immune decoy when the absorbance of the monoclonal antibody is decreased.

In still another aspect, a method for quantitating the decoy antigen in the porcine circovirus 2 (PCV2) antigen using an enzyme-linked immunosorbent assay (ELISA) is disclosed.

With respect to the method for quantitating the decoy antigen in the PCV2 antigen, the ScFV-human Cκ fusion recombinant protein, which specifically binds to the decoy epitope of PCV2, may be used.

With respect to the method for quantitating the decoy antigen in the PCV2 antigen, the ScFV-human Cκ fusion recombinant protein, which specifically binds to the decoy epitope of PCV2, may be C4-1, C4-8, or C4-1 and C4-8.

With respect to the method for quantitating the decoy antigen in PCV2 antigen antibody according to the present invention, the decoy epitope of PCV2 may be the amino acids of STIDYFQPMMKR positioned from the 169^{th} to the 180^{th}.

### Advantageous Effects of the Invention

Accordingly, the monoclonal antibody according to the present invention can make it possible to determine whether the antibody with respect to PCV2 is a neutralizing antibody by a vaccine antigen or an antibody induced by immune decoy.

### BRIEF DESCRIPTION OF THE DRAWINGS

FIG. 1 shows the sequence of the amino acid sequence of a decoy epitope due to a PCV2 infection according to Example 1.

FIG. 2 shows the absorbance of 96 clones randomly selected from the bio-panning using an immune library of a chicken according to Example 4.

FIG. 3 shows the information on the subcloning vector for expressing scFv of the C4-1 and C4-8 clones selected according to Example 5 in a mammalian cell in the form of a human immunoglobulin Cκ fusion protein.

FIG. 4 shows the measurement results of absorbance confirming that the scFv-human Cκ fusion recombinant proteins of the C4-1 and C4-8 selected according to Example 6 specifically binds to the amino acids positioned from the 169^{th} to the 180^{th}.

FIG. 5 shows the measurement results of absorbance confirming the affinity of the scFv-human Cκ fusion recombinant proteins of the C4-1 and C4-8 according to Example 6 to CP-BSA peptide.

FIG. 6 shows the measurement results of absorbance confirming the scFv-human Cκ fusion recombinant proteins of the C4-1 and C4-8 according to Example 7 compete with pig serum for the CP-BSA peptide.

FIG. 7 shows the measurement results of absorbance of the scFv-human Cκ fusion recombinant proteins of the C4-1 and C4-8 according to Example 7 with respect to CP-BSA peptide by a competition ELISA for a group of 20 pigs, which were vaccinated with virus like particle (VLP) of PCV2 (icosahedral), and a group of 20 pigs which were exposed to PCV2 infection due to lack of vaccination.

### DESCRIPTION OF EXEMPLARY EMBODIMENTS

Unless specified otherwise, all the technical and scientific terms used in the present application have the same meaning as that usually understood by an ordinary specialist in the field to which the invention belongs. All the patents, patent applications, patent application publications, Genbank sequences, databases, websites, and other references mentioned over the entire disclosure of the present application, unless known otherwise, are incorporated in their entirety by way of reference. When URL or other such identifiers or addresses are referenced, such identifiers may be changed and particular information on the internet may vary but equivalent information may be discovered by searching through the internet. The reference of the present application proves the availability and pervasiveness of the information made public.

As used herein, the term “antibody” refers to an immunoglobulin or an immunoglobulin fragment, which includes any fragment including at least a part of the variable region of an immunoglobulin molecule, which possesses the specific binding affinity of a full-length immunoglobulin, which is a native or synthesized (e.g., prepared by recombination) in part or entirety. Accordingly, an antibody includes a protein, which has a binding domain homologous to the immunoglobulin antigen-binding domain (antibody binding domain), or substantially the same. The antibody may include a synthetic antibody, an antibody prepared by recombination, a multi-specific antibody, a human antibody, a non-human antibody, a humanized antibody, a chimeric antibody, an intrabody, or a fragment of antibody, but is not limited thereto. For example, the antibody may include an Fab fragment, an Fab′ fragment, an F(ab′)₂ fragment, an Fv fragment, a disulfide-stabilized Fv (dsFv), an Fd fragment, an Fd′ fragment, a single-chain Fv (scFv), a single-chain Fab (scFab), diabody, and anti-idiotypci (anti-Id) antibody, or an antigen-binding fragment thereof.

As used herein, the term “neutralizing antibody” refers to any antibody or an antigen-binding fragment thereof, which binds to a pathogen and thereby prevents the ability of the pathogen that infects cells and/or causes a disease in a subject. Examples of the neutralizing antibody may include those which bind to virus, bacteria, and fungal pathogens. Typically, the neutralizing antibody provided in the present invention binds to the surface of a pathogen. According to the virus classification, the surface protein may be a capsid protein or a viral coated protein.

As used herein, the term “monoclonal antibody” refers to a population of the same antibody in which each individual antibody molecule in the population is the same as those of others. This characteristic is in contrast to the antibody of the polyclonal population of antibodies having multiple different sequences.

As used herein, the term “Fv antibody fragment” refers to a fragment of an antibody which consists of a variable heavy chain (VH) domain and a variable light chain (VL) domain linked by a non-covalent interaction.

As used herein, the term “scFc fragment” refers to an antibody fragment which includes a variable light chain (VL) and a variable heavy chain (VH) domain, covalently-linked in a random sequence by a polypeptide linker. The linker is a length that enables a crosslinking of two variable domains without substantial interference. Examples of the linker include (Gly-Ser)ₙ residues, in which some of the Glu or Lys residues are dispersed over the entire region for the increase of solubility.

As used herein, the term “linker peptide” or “spacer peptide” refers to a short amino acid sequence that connects two polypeptide sequences (or nucleic acids encoding the amino acids). The “polypeptide linker” refers to a short amino acid sequence that connects two polypeptide sequences. Examples of the polypeptide linker include a linker that connects a peptide transfer domain to an antibody, or a linker that connects two antibody chains within a synthetic antibody fragment (e.g., an scFv fragment).

As used herein, the term “polypeptide” refers to two or more amino acids which are covalently-linked. In the present invention, the terms “polypeptide” and “protein” can be used interchangeably.

As used herein, the term “peptide” refers to a polypeptide having a length of about 2 to t 40 amino acids.

As used herein, the term “peptide” refers to an organic compound including an amino group or carboxylic acid group. A polypeptide includes two or more amino acids. For the purpose of the present invention, the amino acids included in the antibody being provided include 20 naturally-occurring amino acids, non-natural amino acids, and amino acid analogs (e.g., an amino acid where α-carbon has a side chain).

As used herein, the term “amino acid residue” refers to an amino acid in the peptide bond which is formed during the chemical digestion (hydrolysis) of a polypeptide. The amino acid residue described in the present invention is generally in the form of an “L” isomer. The residue within the “D” isomer may be replaced with any L-amino acid residue as long as the desired functional property is maintained by the polypeptide. NH₂ represents a free amino group present in the amino terminus of a polypeptide. COOH represents a free carboxyl group present in the carboxyl terminus of a polypeptide.

As used in the present invention, the “property” of a polypeptide, for example an antibody, refers to any property exhibited by a polypeptide, which includes binding specificity, structural configuration or shape, protein stability, resistance to proteolysis, structural stability, thermal resistance, and pH conditions, but is not limited thereto. For example, the change in the binding specificity of an antibody polypeptide may be able to change the binding ability to antigens and/or various activities, for example, affinity or binding affinity or in vivo activity of the polypeptide.

As used in the present invention, the “functional activity” of a polypeptide, for example an antibody, refers to any activity exhibited by a polypeptide. The activities can be determined by experiments. The activity may include antigen-binding, DNA binding, ligand binding or isomerization, and enzyme activity (e.g., an ability to interact with a biomolecule through the kinase activity or proteolysis activity), but is not limited thereto. The activity to an antibody may include an ability to specifically bind to a particular antigen, antigen-binding affinity, binding affinity of an antigen-binding, on-rate, off-rate, effector function (e.g., neutralization or removal of antigens), ability to promote neutralization of virus, and in vivo activities (e.g., ability to prevent pathogenic infection or invasion, promote the removal of pathogen, penetrate particular tissue or body fluid or cell), but is not limited thereto. The activities may be measured in vivo or in vitro using the known measurement methods (e.g., ELISA, flow cytometer, surface plasmon resonance or an equivalent method for measuring on-rate or off-rate, immunohistochemical method, immunofluorescence histology, microscopic observation, cell-based measurement, flow cytometry, and binding analysis (e.g., panning analysis).

As used in the present invention, the terms “oligonucleotide” and “oligo” are used as synonyms. An oligonucleotide is a polynucleotide including a limited length of nucleotides. One of ordinary skill in the art generally considers an oligonucleotide to have a nucleotide length of about 250 or less, typically about 200 or less, and typically about 100 or less. Typically, the oligonucleotide provided in the present invention is a synthetic oligonucleotide. The synthetic oligonucleotide includes a nucleotide with a length shorter than about 20, 30, 40, 50, 60, 70, 80, 90, 100, 110, 120, 130, 140, 150, 160, 170, 180, 190, and 200. Typically, an oligonucleotide is a single-stranded oligonucleotide. The suffix, “mer”, can be used to indicate the length of oligonucleotides, for example, “100-mer” can be used to describe an oligonucleotide which has a 100 nucleotide length.

As used in the present invention, the terms “polynucleotide” and “nucleic acid molecule” represent an oligomer or polymer which includes deoxyribose nucleic acid (DNA) and ribose nucleic acid (RNA), generally a nucleotide or nucleotide derivative of two or more connected with each other by a phosphodiester bond. The polynucleotide includes, for example, nucleotide analogs, or DNA and RNA derivatives which includes a “backbone” binding (e.g., a phosphodiester bond, a phosphoramidate bond, a phosphorothioate bond, a thioester bond, or a peptide bond (peptide nucleic acid). Polynucleotide (peptide nucleic acid) includes single-stranded and/or double-stranded polynucleotide, for example, not only deoxyribose nucleic acid (DNA) and ribose nucleic acid (RNA), but also analogs of any one of RNA or DNA.

As used in the present invention, the term “primer” refers to a nucleic acid molecule which can act as an initiation point for the template-directed nucleic acid synthesis under an appropriate condition (for example, 4 different nucleoside triphosphate and polymerase (e.g., in the presence of DNA polymerase, RNA polymerase, or reverse transcriptase)) in an appropriate buffer and temperature. It may be understood that a particular nucleic acid molecule can be provided as “a probe” and “a primer”. However, a primer has a 3′ hydroxy group for its extension. Primers can be used for various methods, (e.g., polymerase chain reaction (PCR), reverse transcriptase (RT)-PCR, RNA PCR, LCR, multiple PCR, panhandle PCR, capture PCR, expression PCR, 3′ and 5′ RACE, in situ PCR, ligation-mediated PCR, and other amplification protocols).

As used in the present invention, the term “primer pair” refers to, for example, a primer set including a 5′ (upstream) primer which specifically hybridizes with the 5′ end of a sequence to be amplified by PCR, and a 3′ (downstream) primer which specifically hybridizes with a complementary sequence at the 3′ end. Since “primer” is mentioned as a pool of the same nucleic acid molecules, “primer pair” generally refers to a pair of two pools of primers.

As used in the present invention, the term “panning” refers to an affinity-based selection procedure for the separation of a phage which indicates the region, part, or location of a molecule, which has a specificity to a binding partner, for example a capture molecule (e.g., an antigen) or an amino acid or a nucleotide.

As used in the present invention, the term “isolated” or “purified” polypeptide or protein (e.g., an isolated antibody or an antigen-binding fragment thereof) or a biologically active part thereof (e.g., an isolated antigen-binding fragment) substantially has no cellular material from the cell or tissue, from which the protein is derived, or other contaminated proteins, or has no chemical precursor or other chemical materials at the time of chemical synthesis. With regard to preparations, when they are determined by a standard analysis method used by one of ordinary skill in the art for purity measurement (e.g., thin layer chromatography (TLC), gel electrophoresis, and high performance liquid chromatography (HPLC)) and appear to contain no easily detectable impurities, or are pure enough so that additional purification cannot detectably change the physical and chemical properties of a material (e.g., enzyme and biological activities), they may be determined as being not present. The method for purifying compounds for the preparation of a compound with substantial and chemical purity is known to one of ordinary skill in the art. However, the compound with substantial and chemical purity may be a mixture of stereoisomers. In that case, additional purification may increase specific properties of a compound.

As used in the present invention, the singular forms “a”, “an”, and “the” are intended to include the plural forms as well, unless the context clearly indicates otherwise. Accordingly, the description with respect to a polypeptide including “immunoglobulin domain” may include a polypeptide having a single or multiple immunoglobulin domains.

As used in the present invention, the term “or” may be use to refer to “and/or”, unless it is explicitly indicated to represent a selective item, or the selective items are mutually exclusive.

The range and amount used in the present invention may be expressed by way of “about” particular value or range. “About” also includes an exact amount. Therefore, “about 5 amino acids” refers to “about 5 amino acids” and also “5 amino acids”.

As used in the present invention, the term “random” or “randomly” refers to a case where the incident or environment that is to be described hereinbelow may occur or may not occur, and the description include the exemplary case that the incident or environment occurs and the exemplary case that the incident or environment does not occur. For example, the random variation part means that the part is a variation or non-variation.

As used in the present invention, the abbreviations for the protective groups, amino acids, and other compounds shall comply with the IUPAC-IUB committee on the conventional usage, recognized abbreviations, or biochemical nomenclature, unless described otherwise (see Biochem. (1972) 11(9): 1726-1732).

According to an exemplary embodiment, the present invention provides a monoclonal antibody specific to porcine circovirus 2 (PCV2).

In the above exemplary embodiment, the monoclonal antibody specific to PCV2 may be an scFV-human Cκ fusion recombinant protein that specifically binds to a decoy epitope of PCV2.

In the above exemplary embodiment, the monoclonal antibody specific to PCV2 may be C4-1, C4-8, or C4-1 and C4-8 of the ScFV-human Cκ fusion recombinant protein, which specifically binds to a decoy epitope of PCV2.

In the above exemplary embodiment, the decoy epitope of PCV2 may be 12 amino acids positioned from the 169^{th} to the 180^{th}.

In the above exemplary embodiment, the decoy epitope of PCV2 may be a STIDYFQPMMKR peptide.

In the above exemplary embodiment, the monoclonal antibody may be used for analyzing the characteristics of PCV2 using an enzyme-linked immunosorbent assay (ELISA). The method for analyzing the characteristics of the PCV2 antibody using an enzyme-linked immunosorbent assay (ELISA) includes:

performing a competitive reaction between the monoclonal antibody according to the present invention and the antibody in the serum of a subject infected with PCV2 or vaccinated subject;

measuring the absorbance of the monoclonal antibody; and

determining whether the antibody in the serum is a neutralizing antibody or an antibody induced by immune decoy based on the absorbance of the monoclonal antibody. The method for determining whether the antibody in the serum is a neutralizing antibody or an antibody induced by immune decoy may further include determining the antibody as a neutralizing antibody when the absorbance of the monoclonal antibody is constantly maintained; and determining the antibody as an antibody induced by immune decoy when the absorbance of the monoclonal antibody is decreased.

According to another exemplary embodiment, the present invention provides a reagent for diagnosing PMWS, which includes an antigen for diagnosis, a monoclonal antibody for capturing the antigen for diagnosis, a label for detection, a monoclonal antibody to which the label for detection is bound, and a reagent for measuring the activity of the label for detection.

In the above exemplary embodiment, the monoclonal antibody for capturing the antigen for diagnosis may be a monoclonal antibody specific to porcine circovirus 2 (PCV2).

In the above exemplary embodiment, the monoclonal antibody specific to PCV2 may be an ScFV-human Cκ fusion recombinant protein, which specifically binds to a decoy epitope of PCV2.

In the above exemplary embodiment, the monoclonal antibody specific to PCV2 may be C4-1, C4-8, or C4-1 and C4-8 of the ScFV-human Cκ fusion recombinant protein, which specifically binds to a decoy epitope of PCV2.

In the above exemplary embodiment, the decoy epitope of PCV2 may be 12 amino acids positioned from the 169^{th} to the 180^{th}.

According to another exemplary embodiment, the present invention provides a kit for diagnosing PMWS, which includes an antigen for diagnosis, a monoclonal antibody for capturing the antigen for diagnosis, a label for detection, a monoclonal antibody to which the label for detection is bound, and a reagent for measuring the activity of the label for detection.

In the above exemplary embodiment, the monoclonal antibody for capturing the antigen for diagnosis may be a monoclonal antibody specific to porcine circovirus 2 (PCV2).

In the above exemplary embodiment, the monoclonal antibody specific to PCV2 may be an ScFV-human Cκ fusion recombinant protein, which specifically binds to a decoy epitope of PCV2.

In the above exemplary embodiment, the monoclonal antibody specific to PCV2 may be C4-1, C4-8, or C4-1 and C4-8 of the ScFV-human Cκ fusion recombinant protein, which specifically binds to a decoy epitope of PCV2.

In the above exemplary embodiment, the decoy epitope of PCV2 may be 12 amino acids positioned from the 169^{th} to the 180^{th}.

In the above exemplary embodiment, the decoy epitope of PCV2 may be a STIDYFQPMMKR peptide.

Hereinafter, the present invention will be explained in further detail with reference to Examples. However, the present invention should not be limited by these Examples.

### Example 1. Preparation of PCV2 Antigen

1-1. Synthesis of Peptides

Cysteine was added to the end of the 12 amino acids positioned from the 169^{th} to the 180^{th}, which was known as a decoy epitope of porcine circovirus 2 (PCV2), to synthesize a peptide (Peptron, Korea). BSA and KLH were conjugated to the cysteine of the C-terminus and used it as an antigen for screening antibody and the immunity of chickens. The STIDYFQPMMKR peptide was named as CP-BSA or CP-KLH, short for the circovirus peptide.

1-2. PCV2 Recombinant Protein (Monomer)

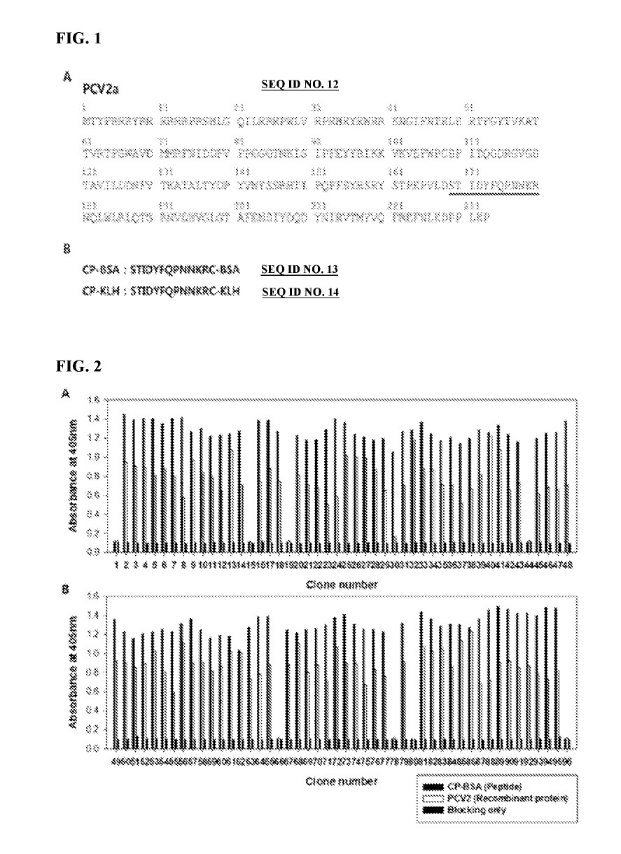

The amino acids at the N-terminus of PCV2 were deleted not to generate an icosahedral structure but to be present in a monomeric form to expose the CP (169 to 180) decoy epitope, thereby enabling a recombinant protein expressed in E. coli to include the amino acid residues of the CP of PCV2 from the 43^{rd} to the 233^{rd} (FIG. 1). The recombinant protein was expressed in E. coli using the method known in the art [see (Pileri E et al., Vet J. 2014, 201(3), 429-32, Comparison of the immunoperoxidase monolayer assay and three commercial ELISAs for detection of antibodies against porcine circovirus type 2)] and purified for use.

1-3. PCV2 Virus Like Particle (VLP, Icosahedral)

PCR was performed using the ORF2 gene of PCV2 virus along with the following primers and each ORF2 gene was subjected to TA cloning into the XL-Topo vector.

TABLE 1

| PCV2-ORF2- | 5′ AAGGATCC- | SEQ ID NO. |
|---|---|---|
| F | ATGACGTATCCAAGGAGGCGTT 3′ | 1 |
| PCV2-a-R | 5′ GCTCTAGA- | SEQ ID NO. |
| | TTAGGGTTTAAGTGGGGGGTCT 3′ | 2 |
| PCV2-b-R | 5′ GCTCTAGA- | SEQ ID NO. |
| | TTAAGGGTTAAGTGGGGGGTCT 3′ | 3 |
| PCV2-d-R | 5′ GCTCTAGA- | SEQ ID NO. |
| | TTAGGGGTTAAGTGGGGGGTCT 3′ | 4 |

After PCV2a-1 was treated with NotI and SpeI restriction enzymes and PCV2d was treated with NotI and BamHI restriction enzymes respectively, a vector containing ORF2 gene and the pFast BacI vector for the expression of baculovirus were reacted with T4 DNA ligase for 2 hours 30 minutes at 1° C. Then, the resultant was transformed into E. coli DH5a, plated on an LB agar containing ampicillin, cultured at 37° C. overnight, and transformed colonies were isolated. The isolated plasmid vectors were subjected to a midi-prep for transfection using the NucleoBond® Xtra Midi Plus. The Sf9 cells obtained from Invitrogen (Lot. No. 1211757) were cultured using Grace's insect medium (10% FBS). The Sf9 cells were cultured in a 6-well plate using Sf-900II media to a concentration of 9×10⁵ cells/well, transfected, and the virus was obtained 3 days thereafter. Each recombinant baculovirus was infected into High5 cells (Invitrogen: Lot. NO. 1179361), which were cultured using Express Five® SFM (L-glutamine 200 mM) media at a concentration of 0.5 MOI, and the PCV2 VLP obtained from the culture broth cultured for 8 days thereafter, was used for tests.

### Example 2. Preparation of Library of Immune Antibody Against PCV2

2-1. Immunization

The peptide KLH conjugate (CP-KLH; 50 μg) synthesized in Example 1, as an antigen, was mixed with phosphate buffered saline (PBS; 750 μL) and cultured at 37° C. for 30 minutes. Then, the resultant was emulsified in an adjuvant of water-in-oil emulsion (RIBI+MPL+TDM+CWS adjuvant, Sigma, St. Louis, Mo., USA) containing detoxified endotoxin (monophosphorylate lipid A species; MPL) and mycobacterial cell wall components (TDW, CWS) in 2% squalene, and then subcutaneously injected to 3 chickens. In the same manner, the chickens were further inoculated 3 weeks thereafter, and subsequently 2 weeks thereafter thereby performing a total of 3 immunizations. The antibody titer of the immunized chickens was determined by an enzyme-linked immunosorbent assay (ELISA) using the horseradish peroxidase (HRP) conjugated anti-chicken IgG (Y) polyclonal antibody (rabbit anti-chicken IgG(Y)-HRP, Millipore corporation, Billeria, Mass., USA) as a secondary antibody.

2-2. Preparation of Single Chain Fv Library of Chicken

Total RNA was extracted from spleen, spleen, bursa, and bone marrow of the immunized chickens of 2-1 using TRI reagent (Invitrogen, Carlsbad, Calif., USA). First strand cDNA was synthesized using oligo-dT primer and Superscript™ III First-Strand Synthesis System (Invitrogen).

A single-chain Fc (scFv) library was prepared using the primers of Table 2 below, which are specific to the heavy chain variable region and light chain variable region of immunoglobulin using the Expand High Fidelity PCR system (Roche Molecular Systems, IN, USA) with respect to the cDNA obtained from the immune system of chickens.

TABLE 2
Vλ Primers

| | | |
|---|---|---|
| CSCVK | GTG GCC CAG GCG GCC CTG ACT CAG CCG TCC TCG | SEQ ID |
| (sense) | GTG TC | NO. 5 |
| CKJo-B | GGA AGA TCT AGA GGA CTG ACC TAG GAC GGT CAG | SEQ ID |
| (reverse) | G | NO. 6 |

V_{H} Primers

| | | |
|---|---|---|
| CSCVHo- | GGT CAG TCC TCT AGA TCT TCC GGC GGT GGT GGC | SEQ ID |
| FL | AGC TCC GGT GGT GGC GGT TCC GCC GTG ACG TTG | NO. 7 |
| (sense) | GAC GAG | |
| CSCG-B | CTG GCC GGC CTG GCC ACT AGT GGA GGA GAC GAT | SEQ ID |
| (reverse) | GAC TTC GGT CC | NO. 8 |

Overlap Extension Primers

| | | |
|---|---|---|
| CSC-F | GAG GAG GAG GAG GAG GAG GTG GCC CAG GCG GCC | SEQ ID |
| (sense) | CTG ACT CAG | NO. 9 |
| CSC-B | GAG GAG GAG GAG GAG GAG GAG CTG GCC GGC CTG | |
| (reverse) | GCC ACT AGT GGA GG | |

In each reaction, 1 μL of cDNA was mixed with 60 pmol of each primer, 10 μL of 10× reaction buffer, 8 μL of 2.5 mM dNTP (Promega, Madison, Wis., USA), 0.5 μL of Tap DNA polymerase and water to a final volume of 100 μL. The PCR reaction was performed for a total of 30 cycles under the following conditions: at 94° C. for 15 sec, at 56° C. for 30 sec, and at 72° C. for 90 sec, and subsequently at 72° C. for 10 min for final extension. The fragment having a size of about 350 bp was electrophoresed by loading on a 1.5% agarose gel and purified using a QIAGEN II Gel Extraction Kit (QIAGEN, Valencia, Calif., USA). The purified PCR product was read at OD 260 nm and quantitated (1 OD unit=50 μg/mL).

In the second PCR, the first VL and VH products were randomly connected by overlap extension PCR. Each PCR reaction was performed by mixing 100 ng of purified VL and VH products, 60 pmol of each primer, 10 μL of 10× reaction buffer, 8 μL of 2.5 mM dNTP, 0.5 μL of Tap DNA polymerase and water to a final volume of 100 μL. The PCR reaction was performed for a total of 25 cycles under the following conditions: at 94° C. for 15 sec, at 56° C. for 30 sec, and at 72° C. for 2 min, and subsequently at 72° C. for 10 min for final extension. The scFv fragment having a size of about 700 bp was electrophoresed by loading on a 1.5% agarose gel and purified using a QIAGEN II Gel Extraction Kit (QIAGEN). The purified PCR product was read at OD 260 nm and quantitated (1 OD unit=50 μg/mL).

2-3. Library Ligation and Transformation

For the cloning of the scFv fragment and pComb3×-SS vector (The Scripps Research Institute, CA, USA), which are PCR products, were cleaved with SfiI restriction enzyme. The purified overlap PCR product (10 μg) was mixed with 360 unit of SfiI (16 units/μg of DNA, Roche Molecular Systems, Pleasanton, Calif., USA), 20 μL of 10× reaction buffer, and water to a final volume of 200 μL. The mixture was cleaved at 50° C. for 8 hours. The scFv fragment with a size of about 700 bp and a vector with a size of 3400 bp were electrophoresed by loading on a 1% agarose gel and purified using a QIAGEN II Gel Extraction Kit (QIAGEN, Valencia, Calif., USA).

The SfiI-cleaved pComb3× vector (1400 ng) and the scFv fragment (700 ng) were mixed with 40 μL of 5× ligase buffer, 10 μL of T4 DNA ligase (Invitrogen, Carlsbad, Calif., USA), and water to a final volume of 200 μL, and ligated by culturing at 16° C. for 16 hours. Then, the resultant was precipitated with ethanol and only the DNA pellet was dissolved in 15 μL of water.

The ligated library sample was transformed into ER2738 (New England Biolabs Inc., Hitchin, Hertfordshine, SG4 OTY, England, UK), an E. coli strain, by electroporation using the Gene pulser (Bio-Rad Laboratories, Hercules, Calif., USA). The cells were mixed in a super broth (SB) medium (5 mL) at 37° C. and cultured with stirring at 250 rpm for 1 hour. Then, 10 mL of SB medium and 3 μL of carbenicillin (100 mg/mL) were added to the culture broth. The culture broth (0.1 μL, 1 μL, and 10 μL) was plated on a Luria broth (LB) agar plate containing carbenicillin (50 μg/mL) and the library size was determined. The culture product was stirred for additional 1 hour and 4.5 μL of carbenicillin (100 mg/mL) was added to the culture broth and was stirred further for additional 1 hour. 2 mL of VCM13 helper phage (>10¹¹ cfu/mL), 183 mL of preheated SB, and 92.5 μL of carbenicillin (100 mg/mL) were added to the culture broth and stirred at a rate of 250 rpm at 37° C. for 2 hours. 280 μL (50 mg/mL) of kanamycin was added to the culture broth and stirred at a rate of 250 rpm at 37° C. overnight. On the next day, the culture broth was centrifuged at 3,000 g, 4° C. using a high speed centrifuge (Beckman, JA-10 rotor). Then, the bacteria pellet was stored for the preparation of phagemid DNA and the supernatant was transferred into a sterile centrifuge bottle. Subsequently, 8 g of polyethylene glycol-8000 (PEG-8000, Sigma) and 6 g of NaCl (Merck) were added thereto, stored on ice for 30 minutes, and the supernatant was centrifuged at 15,000 g, 4° C. for 15 minutes. The supernatant was discarded and the phage pellet was resuspended in Tris-buffer saline (TBS) containing 1% BSA.

### Example 3. Library Panning on Fixed Antigen (Bio-Panning)

Bio-panning was performed using magnetic beads (Dynabeads M-270 Epoxy, Invitrogen). An antigen was coated while rotating/stirring 3 μg of CP-BSA (peptide) and PCV2 recombinant protein (monomer) to the 1×10⁷ beads at room temperature for 20 hours. The coated beads were washed 4 times with PBS, blocked with PBS containing 3% BSA at room temperature for 1 hour, and cultured with the phage-displayed scFv obtained from Example 2-3 at room temperature for 2 hours. To remove the phage, which is not bound to the antigen coated to the beads, the beads were washed with 0.05% Tween 20/PBS, and the bound phage was eluted using 50 μL of 0.1 M glycine-HCl (pH 2.2) and neutralized with 2 M Tris-HCl (pH 9.1). E. coli ER2738 was infected using the phage-containing supernatant and, for the overnight amplification of phage, was rescued using VCSM13 helper phage. Additionally, the culture broth infected with the phage was plated on an LB agar plate containing carbenicillin, and the titer of input and output phages was determined. On the next day, as in Example 1-4, PEG-8000 and NaCl were added thereto to precipitate only phage, and the precipitated phage was used for the next bio-panning.

By repeating the above procedure, panning was performed as follows: using a CP-BSA peptide for the 1^{st} panning, using a PCV2 recombinant protein as an antigen for from the 2^{nd} panning, and alternatively using a peptide and a recombinant protein for from the 3^{rd} to the 7^{th} panning. Additionally, with respect to the washing step, in the 1^{st} panning, the number of washing was gradually increased since the 1^{st} wash and performed the 10^{th} wash in the 7^{th} panning, thereby allowing selection and enrichment of phages with high affinity.

### Example 4. Selection of Clones by Phage ELISA

For the analysis of clones selected from bio-panning, an ELISA was performed to confirm whether the randomly selected individual phage-displayed scFv clones simultaneously have binding affinity to PCV2 CP-BSA peptide and PCV2 recombinant protein (monomer).

CP-BSA (peptide) and a PCV2 recombinant protein (monomer) were diluted in 0.1 M NaHCO₃ buffer, coated into a 96-well microtiter plate at a concentration of 100 ng/well at 4° C. for 16 hours, and blocked with 3% BSA/PBS at 37° C. for 1 hour. Then, the phage supernatant was mixed equally with 6% BSA/PBS and cultured at 37° C. for 2 hours. The culture broth was washed with 0.05% Tween 20/PBS, and the HRP conjugated anti-M13 antibody (a-M13-HRP, Pierce Chemical Co, Rockford, Ill., USA) was diluted in a 1:5000 ratio, added into the plate in an amount of 50 μL, and cultured at 37° C. for 1 hour. Upon completion of cultivation and washing, for the color reaction, 2,2′-azino-bis(3-ethylbenzothiazoline-6-sulphonic acid) (ABTS, Amresco, Solon, Ohio, USA) at a concentration of 1 μg/mL and 0.1% H₂O₂ were added into each well in the 0.05 M citrate buffer, and developed color, and the absorbance was measured at 405 nm. The results are shown in FIG. 2.

FIG. 2a and FIG. 2b respectively show the analysis result of 48 clones in output phage of the 6^{th} and 7^{th} panning, respectively. Among the clones which simultaneously bind to the CP-BSA peptide and the PCV2 recombinant protein, the gene sequences of 10 clones with highest absorbance were analyzed, and thereby scFv clones with a total of 3 different kinds of sequences were obtained and the subsequent C4-1 and C4-8 clones were used.

### Example 5. Preparation of Recombinant Anti-PCV2 scFv-Human Cκ Fusion Protein

5-1. Subcloning of Anti-PCV2 scFv Using a Mammalian Expression Vector (scFv-Cκ)

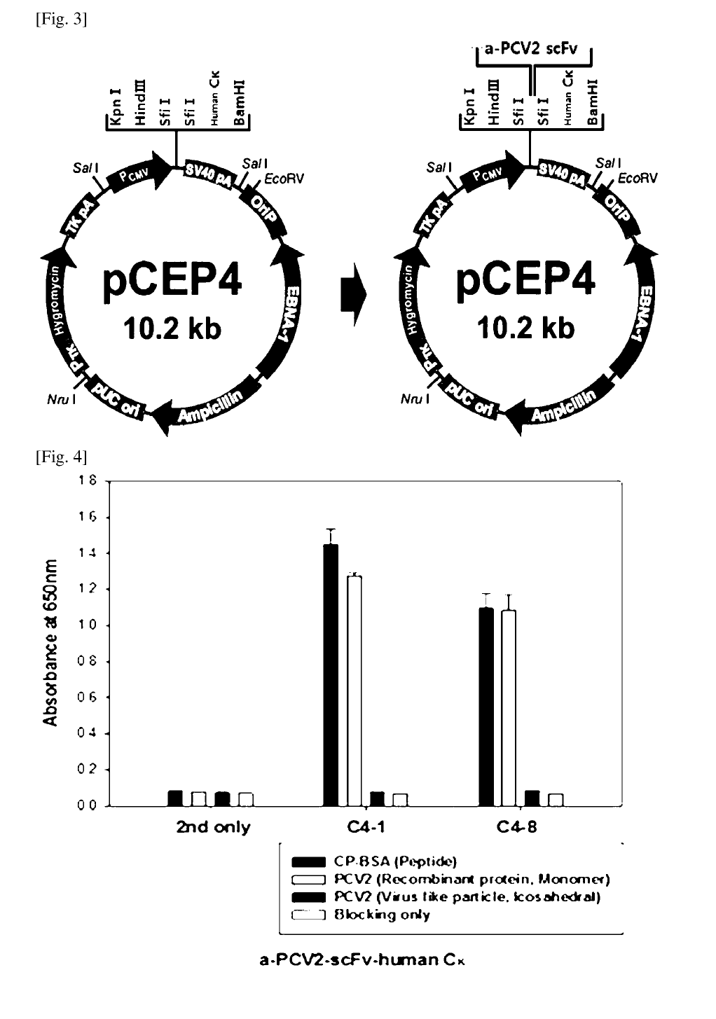

The mammalian expression vector pCEP4 (Invitorgen), in which restriction sites are modified for easy cloning, was inserted with a gene encoding human immunoglobulin Cκ by HindIII and XhoI (New England Biolabs, UK) restriction enzymes. The gene encoding the anti-PCV2 scFv was subcloned from the pComb3× vector into the 5′ end of human Cκ region by two SfiI restriction sites (FIG. 3)

5-2. Transfection and Protein Purification

For the expression and purification of pCEP4-anti PCV2 scFv-human Cκ into a protein form, a mammalian transfection and overexpression system were used. Per mL of the culture volume, 2 μg of a mammalian expression vector and 4 μg of polyethyleneimine (PEI, Polysciences, Warrington, Pa., USA) were mixed with 150 mM NaCl (Merck) corresponding to a 1/10 of the cell culture volume, and placed at room temperature for 15 minutes. The mixture was added to a mammalian cell HEK293F (2×10⁶ cells/mL, Invitrogen) and cultured in a Freestyle™ 293 expression medium containing 100 U/mL of penicillin and streptomycin in conditions of at 37° C., 7% CO₂, and stirring at 135 rpm for 6 days. The cell culture broth was centrifuged and, for the purification of only the fusion protein in the form of anti-PCV2 scFv-Cκ expressed from the supernatant, an affinity gel chromatography using the Kappaselect (GE Healthcare Bio science, Sweden) was used.

### Example 6. Measurement of Binding Ability of scFv-Cκ Fusion Protein

6-1. Confirmation of PCV2-Binding Region of Anti-PCV2-scFv-Cκ Fusion Protein

To confirm whether the antibody of the scFv-Cκ fusion protein produced in Example 5 exhibits the binding ability only when the amino acids positioned from the 169^{th} to the 180^{th}, which is expected to be as the CP-BSA peptide and epitope, is in an exposed form, an enzyme-linked immunosorbent assay (ELISA) was performed. As the type of the antigen to be coated, CP-BSA peptide, PCV2 recombinant protein (monomer), and PCV2 virus like particle (VLP, icosahedral) were diluted in 0.1 M NaHCO₃, and added to a 96-well microtiter plate at a concentration of 100 ng/well, and coated at 4° C. for 16 hours. The anti-PCV2 scFv-Cκ fusion protein was diluted in 3% BSA/PBS at a concentration of 1 μg/mL and added to each well in an amount of 50 μL and reacted for 2 hours. Upon completion of the cultivation, the culture broth was washed with 0.05% Tween 20/PBS, and HRP conjugated anti-human Cκ antibody (Goat anti-human Cκ-HRP, Abcam, Cambridge, UK) was diluted in a 1:5000 ratio and added to each well at a concentration of 50 μL/well, cultured for 1 hour, washed with tetramethylbenzidine (TMB, Gendepot, Barker, Tex., USA), allowed to develop a color, and the absorbance was measured at the wavelength of 650 nm. The results are shown in FIG. 4. The experiment was performed in duplicate and the graph was drawn using the mean value and the error range was indicated by means of standard deviation.

As can be seen from FIG. 4, from the observation that the anti-PCV2-scFv-Cκ fusion protein can bind only in the form of the CP-BSA peptide and PCV2 recombinant protein (monomer) but not in the PCV2 virus like particle (VLP, icosahedral) where the epitope is hidden, it was confirmed that the binding region of the PCV2 is the amino acids positioned from the 169^{th} to the 180^{th}, which is the PCV2 decoy epitope.

6-2. Measurement of Binding Ability of scFv-Cκ Fusion Protein

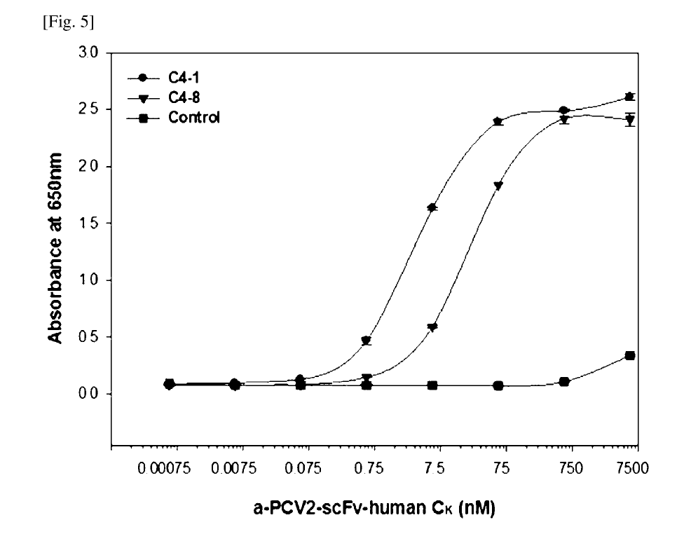

To measure the binding ability of the scFv-Cκ fusion protein produced in Example 5 to the CP-BSA peptide, an ELISA was performed. CP-BSA peptide was diluted in 0.1 M NaHCO₃, and subjected to a 1:10 serial dilution starting from 7500 nM (the concentration where the scFv-Cκ fusion protein is included in 50 μL a 100-fold moles of the epitopes of the CP-BSA peptide) to 0.00075 nM by adding at a concentration of 8 points to a 96-well coated plate at a concentration of 25 ng/well at 4° C. for 16 hours, and reacted for 2 hours. As the types of the scFv-Cκ fusion proteins to be added, the C4-1 and C4-8 clones, which have the binding affinity for the CP-BSA peptide were used, and the Control-scFv-Cκ, which does not bind to the CP-BSA peptide, was used as a negative control. Then, the absorbance was measured in the same manner as in Example 6-1, and the results are shown in FIG. 5. The experiment was performed in duplicate, and the graph was drawn using the mean value and the error range was indicated by means of standard deviation.

As can be seen from FIG. 4, it was confirmed that the C4-1 and C4-8 clones have excellent binding affinity for the CP-BSA peptide.

### Example 7. Anti-PCV2-scFv-Cκ Fusion Protein and Competition ELISA Between Porcine Sera

7-1. Competition ELISA Between Porcine Sera According to the Concentration of Anti-PCV2-scFv-Cκ Fusion Protein

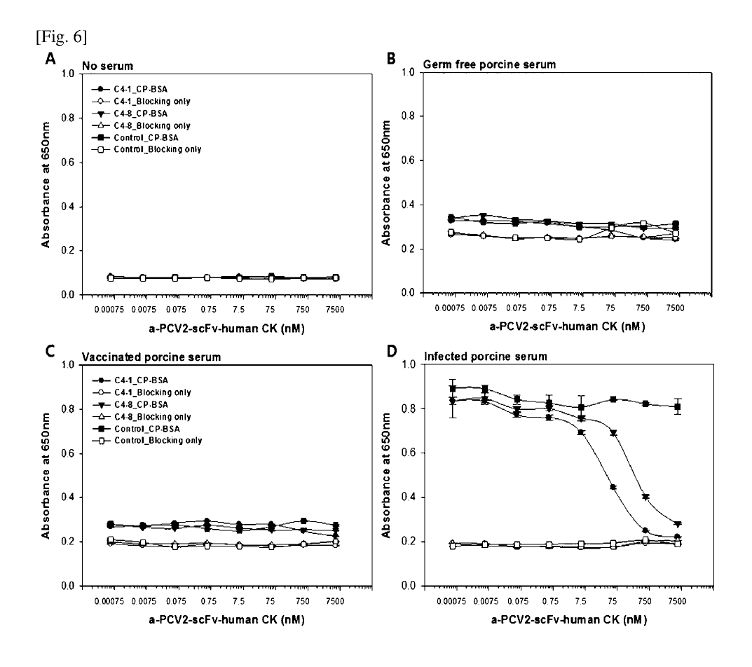

Whether the competition effect between the porcine antibody present in the serum produced with respect to the amino acids positioned from the 169^{th} to the 180^{th}, which is the region to be exposed by infection, and the anti-PCV2-scFv-Cκ generates a concentration gradient according to the amount was confirmed using the antibody in the form of the anti-PCV2-scFv-Cκ fusion protein produced in Example 4. The point where the signal changes most rapidly was set by a 1:2 ratio dilution of the amount of the antigen to be coated through a preliminary experiment, and diluted in 0.1 M NaHCO₃ at a concentration of 25 ng/well, and coated to a 96-well microtiter plate at 4° C. for 16 hours. On the next day, each serum of germ-free pigs (Seoul national university, Korea), vaccinated pigs, and infected pigs was diluted in 3% BSA/PBS at a 1:50 ratio, and the anti-PCV2-scFv-Cκ and negative control scFv-Cκ were prepared by a 1:10 serial dilution starting from 15000 nM to 0.00015 nM by adding at a concentration of 8 points, and the sera and the anti-PCV2-scFv-Cκ were mixed at an equal volume of 25 μL. As the final concentration, with respect to the serum, the 1:100 anti-PCV2-scFv-Cκ was added at a concentration of 8 points from 7500 nM to 0.00075 nM and reacted for 2 hours. The plate was washed with 0.05% Tween 20/PBS and, the HRP conjugated anti-swine immunoglobulin antibody (goat anti-swine IgG-HRP, Santacruz, Calif., USA), as a secondary antibody, was diluted in a 1:4000 ratio and used at a concentration of 50 μL/well. 1 hour thereafter, tetramethylbenzidine (TMB, Gendepot, Barker, Tex., USA) was added thereto to develop a color and the absorbance was measured at the wavelength of 650 nm. The results are shown in FIG. 6. For A, one in which no porcine serum added was used, germ-free porcine serum was used for B, vaccinated porcine serum for C, and infected porcine serum for D. The experiment was performed in duplicate, and the graph was drawn using the mean value and the error range was indicated by means of standard deviation.

As can be seen from FIG. 6, it was confirmed that although the scFv-Cκ fusion protein is added in an excess amount in the serum inoculated with a germ-free serum or vaccinated serum there occurs no competition and is maintained at a low signal because these sera do not produce the antibody to the CP epitope. In contrast, in the case of infected pigs, they produce an antibody to the CP epitope, and thus there occurs a competition with the scFv-Cκ fusion protein, and there was a result that as the concentration of the scFv-Cκ being added increased the signal to the antibody present in the serum was decreased by the competition.

7-2. Anti-PCV2-scFv-Cκ Fusion Protein and Competition ELISA Between Porcine Sera

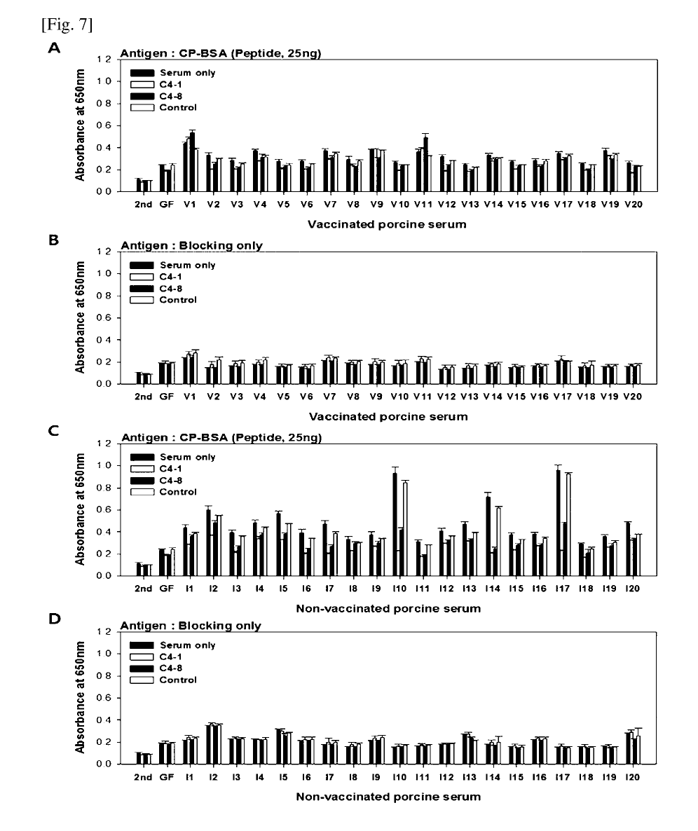

The experiment was performed in the same manner as in Example 7-1. The amount of antigen to be coated was set at 25 ng/well, and the final concentration of the primary antibody was set at a 1:100 ratio, and the anti-PCV2-scFv-Cκ fusion protein at 750 nM. Experiments were performed using a total of 41 pigs; i.e., a single germ-free pig as the control, and the sera from a group of 20 vaccinated pigs and the sera from a group of 20 pigs suspected of infection due to lack of vaccination. The results are shown in FIG. 7. FIG. 7A shows the result of the experiment where the CP-BSA peptide was used as an antigen and FIG. 7B shows the result of the experiment where the sera of a group of vaccinated pigs were used as the blocking control. FIG. 7C shows the result of the experiment where the CP-BSA peptide was used as an antigen and FIG. 7D shows the result of the experiment where the sera of a group of unvaccinated pigs were used as the blocking control. The experiment was performed in triplicate, and the graph was drawn using the mean value and the error range was indicated by means of standard deviation.

As can be seen from FIG. 7, it was confirmed that the sera of the group of vaccinated pigs showed almost no change in the signal, but the sera of the group of unvaccinated pigs showed a decrease in the signal because an antibody to the amino acids positioned from the 169^{th} to the 180^{th} was formed by the infection thus causing a competition with the anti-PCV2-scFv-Cκ fusion protein.

### INDUSTRIAL APPLICABILITY

The monoclonal antibody according to the present invention can make it possible to determine whether the antibody against PCV2 is a neutralizing antibody by a vaccine antigen or an antibody induced by immune decoy.

1. A monoclonal antibody specific to porcine circovirus 2 (PCV2), wherein the monoclonal antibody is scFV-human Cκ fusion recombinant protein that specifically binds to a decoy epitope of PCV2.

2. The monoclonal antibody of claim 1, wherein the ScFV-human Cκ fusion recombinant protein, which specifically binds to a decoy epitope of PCV2, is C4-1, C4-8, or C4-1 and C4-8.

3. The monoclonal antibody of claim 1, wherein the decoy epitope of the PCV2 is 12 amino acids positioned from the 169^{th} to the 180^{th}.

4. The monoclonal antibody of claim 3, wherein the amino acids are STIDYFQPNNKR (SEQ ID NO: 11).

5. A reagent for diagnosing post-weaning multi-systemic wasting syndrome (PMWS), comprising an antigen for diagnosis, a monoclonal antibody for capturing the antigen for diagnosis, a label for detection, a monoclonal antibody to which the label for detection is bound, and a reagent for measuring the activity of the label for detection, wherein the monoclonal antibody for capturing the antigen for diagnosis is an scFV-human Cκ fusion recombinant protein which specifically binds to the decoy epitope of porcine circovirus 2 (PCV2).

6. The PMWS reagent of claim 5, wherein the ScFV-human Cκ fusion recombinant protein, which specifically binds to a decoy epitope of PCV2, is C4-1, C4-8, or C4-1 and C4-8.

7. The PMWS reagent of claim 5, wherein the decoy epitope of the PCV2 is the amino acids of STIDYFQPNNKR (SEQ ID NO: 11) positioned from the 169^{th} to the 180^{th}.

8. A method for analyzing the characteristics of porcine circovirus 2 (PCV2) antibody using an enzyme-linked immunosorbent assay (ELISA), comprising:
performing a competitive reaction between the monoclonal antibody according to any one of claims 1 to 4 and the antibody in the serum of a subject infected with PCV2 or vaccinated subject;
measuring the absorbance of the monoclonal antibody; and
determining whether the antibody in the serum is a neutralizing antibody or an antibody induced by immune decoy based on the absorbance of the monoclonal antibody.

9. The method of claim 8, further comprising determining the antibody as a neutralizing antibody when the absorbance of the monoclonal antibody is constantly maintained.

10. The method of claim 8, further comprising determining the antibody as an antibody induced by immune decoy when the absorbance of the monoclonal antibody is decreased.

11. A method for quantitating the decoy antigen in the porcine circovirus 2 (PCV2) antigen using an enzyme-linked immunosorbent assay (ELISA), wherein scFV-human Cκ fusion recombinant protein, which specifically binds to a decoy epitope of PCV2, is used as a PCV2-specific monoclonal antibody.

12. The method of claim 11, wherein the ScFV-human Cκ fusion recombinant protein, which specifically binds to a decoy epitope of PCV2, is C4-1, C4-8, or C4-1 and C4-8.

13. The method of claim 11, wherein the decoy epitope of PCV2 is the amino acids of STIDYFQPNNKR (SEQ ID NO: 11) positioned from the 169^{th} to the 180^{th}.


SEQUENCE LISTING (10 sequences)

SEQ ID NO: 1 - DNA, 30 bp, Artificial Sequence
  PRIMER
aaggatccatgacgtatccaaggaggcgtt

SEQ ID NO: 2 - DNA, 30 bp, Artificial Sequence
  PRIMER
gctctagattagggtttaagtggggggtct

SEQ ID NO: 3 - DNA, 30 bp, Artificial Sequence
  PRIMER
gctctagattaagggttaagtggggggtct

SEQ ID NO: 4 - DNA, 30 bp, Artificial Sequence
  PRIMER
gctctagattaggggttaagtggggggtct

SEQ ID NO: 5 - DNA, 38 bp, Artificial Sequence
  PRIMER
gtggcccaggcggccctgactcagccgtcctcggtgtc

SEQ ID NO: 6 - DNA, 34 bp, Artificial Sequence
  PRIMER
ggaagatctagaggactgacctaggacggtcagg

SEQ ID NO: 7 - DNA, 72 bp, Artificial Sequence
  PRIMER
ggtcagtcctctagatcttccggcggtggtggcagctccggtggtggcggttccgccgtgacgttggacgag

SEQ ID NO: 8 - DNA, 44 bp, Artificial Sequence
  PRIMER
ctggccggcctggccactagtggaggagacgatgacttcggtcc

SEQ ID NO: 9 - DNA, 42 bp, Artificial Sequence
  PRIMER
gaggaggaggaggaggaggtggcccaggcggccctgactcag

SEQ ID NO: 10 - DNA, 38 bp, Artificial Sequence
  PRIMER
gaggaggaggaggaggaggagctggccggcctggccac